(19) United States
(12) Patent Application Publication
(10) Pub. No.: US 2023/0329908 A1
(43) Pub. Date: Oct. 19, 2023

(54) FLUIDICS CONTROL FOR MICROSURGICAL DEVICES

(71) Applicant: MICROSURGICAL TECHNOLOGY, INC., Redmond, WA (US)

(72) Inventors: Dave SCOTT, Bothell, WA (US); Haley HUNT, Issaquah, WA (US)

(73) Assignee: MICROSURGICAL TECHNOLOGY, INC., Redmond, WA (US)

(21) Appl. No.: 18/026,941

(22) PCT Filed: Sep. 17, 2021
(86) PCT No.: PCT/US2021/050789

§ 371 Date: Mar. 17, 2023

Related U.S. Application Data
(60) Provisional application No. 63/080,776, filed on Sep. 20, 2020.

(51) Int. Cl. A61F 9/007; A61M 5/142; A61M 1/00

(52) U.S. Cl. CPC A61F 9/00736; A61M 5/142; A61M 1/774; A61M 2210/0612; A61M 2005/14208

(57) ABSTRACT

A microsurgical device contains a fluidics control containing multiple controlled fluidics paths that enable bi-directional flow and fluid separation or mixing. The fluidics control has a housing including a separable end piece. The housing may serve as a mixing chamber that enables mixing multiple fluids or drugs or diluting a concentrated drug to provide varying drug concentration. The end piece may include one or more ports that may be connected to an aspiration pump or variable substance chambers through plastic tubing. The end piece may be rotationally or frictionally connected to the housing and partially enclosed by the housing. The microsurgical device further includes a fluid control element configured to regulate the fluid flow into or out of the device, or a combination thereof.

### RELATED APPLICATION

This application claims priority benefit to U.S. Provisional Application No. 63/080,777, filed Sep. 20, 2020, which is fully incorporated herein by reference for all purposes.

### FIELD OF THE INVENTION

The present invention relates generally to the fields of medicine and engineering and more particularly to systems and methods for mixing and delivering fluids during eye surgery.

### BACKGROUND

Glaucoma is a leading cause of blindness in the United States. The loss of vision in glaucoma patients is typically progressive and may be due, at least in part, to compression of the vasculature of the retina and optic nerve as a result of increased intraocular pressure. It is generally accepted that reducing intraocular pressure, through the use of drugs and/or surgery, can significantly reduce glaucomatous progression in patients who suffer from normal-tension glaucoma and can virtually halt glaucomatous progression in patients who suffer from primary open-angle glaucoma with elevated intraocular pressures. Furthermore, it is generally acknowledged that lowering intraocular pressure in glaucoma patients can prevent or lessen the irreversible glaucoma-associated destruction of optic nerve fibers and the resultant irreversible vision loss.

Surgical therapy for open-angle glaucoma consists of laser trabeculoplasty, trabeculotomy/goniotomy, and implantation of aqueous shunts. Trabeculotomy/goniotomy and other mechanical disruptions of the trabecular meshwork, such as trabeculopuncture, goniophotoablation, laser trabecular ablation, and goniocurretage are all major operations that are widely used. Trabeculotomy and goniotomy are simple and directed techniques of microsurgical dissection of the trabecular meshwork. These techniques are augmented with topically applied anticancer drugs, such as 5-flurouracil or mitomycin-C, to decrease scarring and increase the likelihood of surgical success.

The introduction of drugs during trabeculotomy/goniotomy operations may be problematic if they require either the removal of one of the tools being used in a given procedure or an additional incision in the eye. Either of these options results in increased surgical time and potential complications. Additionally, repeated tool insertions and removals may be necessary if additional substance is needed. More manipulation of tools increases the risk of wound tearing which can lead to an undesirable astigmatic effect, infection, and wound leak.

Furthermore, if the device only makes a single cut (like a classical goniotomy knife used for surgical management of pediatric glaucoma), all of the trabecular meshwork tissue remains behind. For adults, the outcomes for such a procedure may be prone to having the access of fluid drainage via the collector channels in Schlemm's canal become blocked over time as this remnant tissue clogs such access for fluid outflow via the collector channels. Thus, proper removal of a strip of trabecular meshwork greatly enhances the safety of such a procedure.

There remains a need in the art for the development of a microsurgical device that may be used in a multiple-port mode to allow for fluid insertion or removal without the need to introduce a separate cannula.

The need also exists for such improved device to be simple in construction, efficient in operation, and economical to manufacture.

### SUMMARY OF THE INVENTION

In view of the foregoing, an object of the present invention is to address the need for multiple controlled fluid paths by providing a fluidics control for use with a microsurgical device. A still further object of the present invention is to provide a microsurgical device with a variable capacity in infusion or aspiration to allow multiple fluids to be transferred simultaneously through a small volume in micro-incisional glaucoma surgery.

The present disclosure is directed to systems and methods for transferring a substance (e.g., a fluid, tissue, drug, etc.) into or out of an eye during an operation without requiring a separate incision to be made or without requiring introduction of a separate cannula into the eye. For example, a user may perform a procedure that includes removal of trabecular meshwork (TM) from the eye using a goniotomy probe. The same goniotomy probe may also introduce a substance into the eye or remove tissue, fluid or other debris out of the eye. Accordingly, a surgeon or other users may be able to cut and remove TM and inject an amount of the substance into the anterior chamber of the eye of the patient using the same tool.

Depending on the implementation, the microsurgical device may be coupled to an aspiration pump and/or one or more substance chambers. During an aspiration process, the microsurgical probe may aspirate TM tissue, blood, fluid or other debris from the eye during or post-operation. During an irrigation process, the microsurgical probe may enable a surgeon to controllably deliver a desired amount of the substance into the surgical site without removing the microsurgical device from the site or introducing additional cannulas.

Disclosed herein is a fluidics control for use with a handheld microsurgical device to enable bi-directional flow and fluid separation or mixing. In a preferred embodiment, the fluidics control has a housing including a separable end piece. The housing may serve as a mixing chamber that enables mixing multiple fluids or drugs or diluting a concentrated drug to provide varying drug concentration. The end piece may include one or more ports that may be connected to an aspiration pump or variable substance chambers through plastic tubing. The end piece may be rotationally or frictionally connected to the housing and partially enclosed by the housing. In other embodiments, the housing and the end piece may be made in one integrated unit.

The system may further include a fluid control element for variably controlling the flow of fluid through the handheld microsurgical device. The fluid control element is configured to regulate fluid flow into or out of the fluidics control. The fluid control element may also allow for bolus or continuous delivery and enable the measured delivery of drug at independent rates.

In accordance with one aspect of the present invention, a handheld microsurgical device with a fluidics control is provided. An exemplary handheld microsurgical device comprises a handle, a shaft, a distal member or foot on a distal end of the shaft and a fluidics control. The handle includes a body and a lumen formed in the body, and the handle is in fluid communication with the fluid source as well as the aspiration pump. The handle may further include a control element that enables selective control of fluid and adjustment of a flow rate. In one embodiment, the control element includes a switch mounted on the outside surface of the handle with on/off, variable speed, and forward/reverse controls. The shaft comprises an inner tube and an outer tube, wherein the inner tube extends out of and beyond the distal end of the outer tube. The distal member is insertable into Schlemm's canal of an eye and thereafter advanceable through Schlemm's canal such that trabecular meshwork tissue will be cut and/or removed. In some embodiments, the device may optionally include lumens, opening or ports and associated connectors for irrigating fluid and/or aspirating fluid and/or tissue or other debris from the eye.

In accordance with one embodiment, a fluidics control is disposed partially within the handle body. The housing of the fluidics control is rotationally or frictionally affixed to the handle body. The fluidics control is situated between the distal end of the handle body and the proximal end of the shaft. The inner tube of the shaft may have a fluid connection with the housing. Alternatively, the inner tube of the shaft may bypass the housing and be fluidically connected to a port that is coupled to an aspiration pump. The outer tube is fluidically connected to the housing.

The fluidics control of a microsurgical device allows multiple fluids to be transferred (e.g., delivered or removed) simultaneously through a small volume, which is specifically beneficial in micro-incisional glaucoma surgery. The fluidics control enables excision of tissue and capture of material into body of instrument as well as enables the connection of multiple fluids paths, including the potential for localized delivery of drugs.

In view of the foregoing, other aspects, features, details, utilities, and advantages of the disclosed embodiments will be apparent from the following description and claims as well as the accompanying drawings.

### BRIEF DESCRIPTION OF THE DRAWINGS

The following detailed description and examples are provided for the purpose of non-exhaustively describing some, but not necessarily all, examples or embodiments of the invention, and shall not limit the scope of the invention in any way.

FIG. 1 shows one embodiment of a handheld microsurgical device.

FIG. 2 is an enlarged view of a distal portion of the device of FIG. 1.

FIG. 3 is a perspective view of a fluidics control.

FIG. 4 shows a sectional view of a fluidics control according to a first embodiment.

FIG. 5 shows a sectional view of a fluidics control according to a second embodiment.

FIG. 6A shows a top view of the wound sealing sleeve and FIG. 6B shows a sectional view of the wound sealing sleeve shown in FIG. 6A.

FIG. 7 shows a sectional view of a fluidics control according to a third embodiment.

FIG. 8A depicts a perspective view of an exemplary control element that enable on/off, variable speed, and forward/reverse fluid flow controls; FIG. 8B depicts a sectional view of an exemplary switch in an open position; and FIG. 8C shows a sectional view of an exemplary switch in a closed position.

### DETAILED DESCRIPTION OF THE EMBODIMENTS

It should be understood that this invention is not limited to the particular apparatus, methodology, protocols, and systems, etc., described herein and as such may vary. The terminology used herein is for the purpose of describing particular embodiments only and is not intended to limit the scope of the present invention, which is defined solely by the claims.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

The present disclosure is directed to systems and methods for transferring a substance (e.g., a fluid, tissue, drug, etc.) into or out of an eye during an operation without requiring a separate incision to be made and without requiring introduction of a separate cannula into the eye. To illustrate, several exemplary embodiments are described in detail herein. The systems and methods described can be utilized in other contexts.

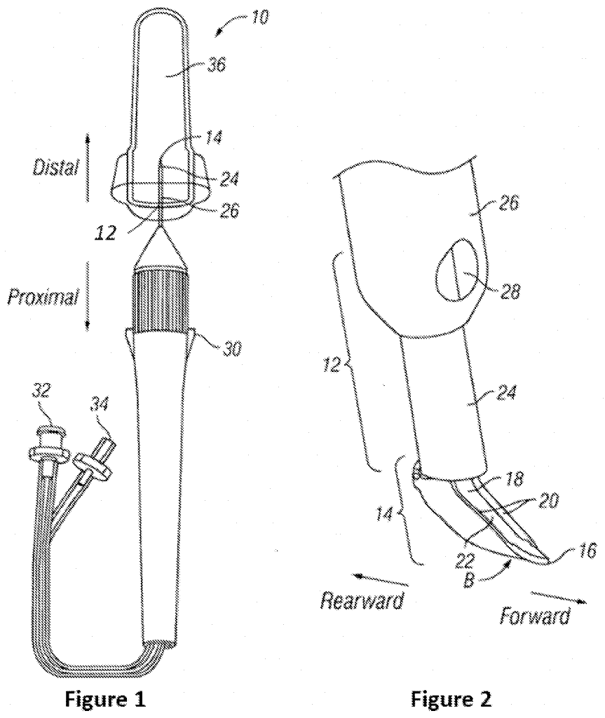

In some examples, a substance may be delivered into or out of an eye through a lumen of an elongate portion of a microsurgical device, such as a goniotomy probe. FIG. 1 depicts one embodiment of a goniotomy device as illustrated in WO 2018/151808, the entirety of which is expressly incorporated herein by reference. This handheld device 10 generally comprises a handle 30, a shaft 12 that extends distally from the handle 30 and a distal member 14 (which is alternatively referred to herein as a “foot”) on the distal end of the shaft 12. The handle includes a body and a lumen formed in the body, and the handle is in fluid communication with the fluid source as well as the aspiration pump. The handle may further include a control mean that enables selective control of fluid and adjustment of a flow rate. In one embodiment, the control mean includes a switch mounted on the outside surface of the handle 30 with on/off, variable speed, and forward/reverse controls. The shaft 12 comprises an inner tube 24 and an outer tube 26. The inner tube 24 extends out of and beyond the distal end of the outer tube 26.

Referring to FIG. 1, the device 10 may include lumens, outlets and connectors for infusion of irrigation fluid into and/or aspiration of fluid/matter. In one embodiment, the device 10 includes tubular infusion and aspiration connectors 32, 34 for connecting sources of irrigation fluid and aspiration to the desired irrigation/aspiration lumens of the shaft 12. More specifically, in the particular non-limiting example shown, tubular connector 32 is configured for connection to a source of irrigation fluid and communicates with the annular lumen that extends through the outer tube around the outer surface of the inner tube 24, thereby facilitating infusion of irrigation fluid through the outer tube 26 and out of opening 28. Tubular connector 34 is configured for connection to an aspiration pump or suction source to facilitate aspiration of fluid and or other matter, into the open distal end of inner tube 24, through the lumen of inner tube 24 and out of tubular connector 34.

FIG. 2 depicts an enlarged view of a distal portion of the device. The inner tube 24 has a smaller outer diameter than an inner diameter of the outer tube 26 such that the inner tube 24 is positioned within the outer tube 26, for example, substantially coaxial therewith. The inner surface of the outer tube 26 is spaced apart from the outer surface of the inner tube 24 thereby defining an annular lumen which communicates with outlet port 28. The distal end of the outer tube 26 tapers down and is in sealing contact with the outer surface of the protruding inner tube 24. The opening 28 is positioned slightly above the location where the inner tube 24 exits the distal end of the outer tube 26.

Referring to FIG. 2, the distal member 14 has a bottom surface B, right and left upwardly extending side walls 22 and a cavity or open area 18 between the side walls 22 and rearward of the forward tip 16. The forward tip 16 may be tapered to a blunt point as shown. Edges 20 form the sides of the forward tip 16 and transition in orientation as they progress in the rearward direction to form spaced-apart, upwardly-sloping top surfaces of the sidewalls 22. All or portions of the upwardly sloping regions of edges 20 may be sharpened, beveled, serrated, or otherwise configured to form cutting regions 21 that facilitate cutting of tissue as it advances over those edges 20. Also, the side walls 22 and/or edges 20 may be non-parallel such that the space 18 between the sidewalls 22 and/or edges 20 becomes wider as it progresses in the rearward direction. This optional widening or non-parallelism of the side walls 22 and/or edges 20 may serve to transverse stretch or transversely tighten tissue as it advances up the progressively-widening, upwardly-sloping regions of edges 20.

In the non-limiting example shown, the upper surface of the distal member is generally trough-shaped (e.g., U-shaped), with the first and second inclined side walls 22 onto which are formed tissue-severing edges or blades 20. These edges 20 are spaced apart, as shown.

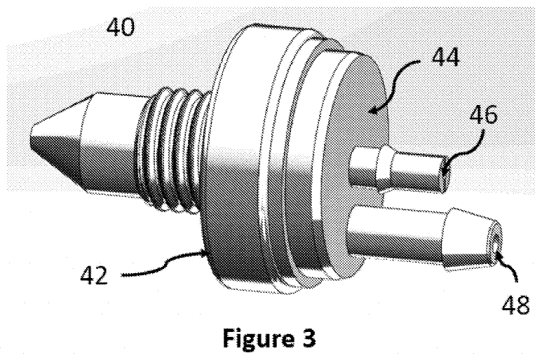

FIG. 3 shows a perspective view of a fluidics control 40 according to one embodiment of the present invention. The fluidics control 40 has a housing 42 and an end piece 44. The housing 42 may include a mixing chamber that enables mixing multiple fluids or drugs or diluting a concentrated drug to provide varying drug concentration. The end piece may include a port 46 that is connected to an aspiration pump through plastic tubing. The end piece may further include one or more ports 48 that are connected to variable substance chambers through plastic tubing. Any port at the end piece may have barbed fittings that allow direct connection of the tubes without any glue. In some embodiments, the housing 42 and the end piece 44 are separable. The end piece may be rotationally or frictionally connected to the housing and partially enclosed by the housing. In other embodiments, the fluidics control 40 is a one-piece manifold that contains a housing 42 and an end piece 44. The fluidics control 40 may be made of stainless steel, aluminum, or various types of metal or plastic materials.

The fluidics control 40 may be disposed partially within the handle body. The housing of the fluidics control is rotationally or frictionally affixed to the handle body. The fluidics control is situated between the distal end of the handle body and the proximal end of the shaft. The inner tube of the shaft may have a fluid connection with the housing. Alternatively, the inner tube of the shaft may bypass the housing and is fluidically connected to a port that is coupled to an aspiration pump. The outer tube is fluidically connected to the housing.

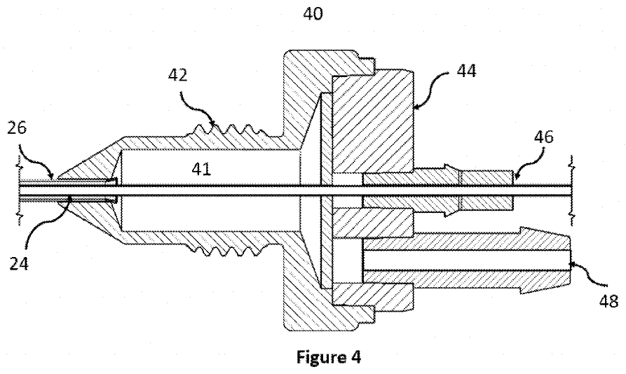

FIG. 4 is a sectional view of the fluidics control 40 according to one embodiment. The housing 42 contains a mixing chamber 41 capable of mixing two or more fluids to produce one or more concentrations of a drug and/or a mixture of several substances. The distal end of the mixing chamber 41 is fluidically connected to the annular lumen of the outer tube 26. The proximal end of the mixing chamber 41 is fluidically connected to a port 48 disposed at the end piece 44. The port 48 may connect to a substance chamber through plastic tubing. It is contemplated that the end piece 44 may include a plurality of ports for connecting to variable substance chambers. The end piece 44 may include a center aspiration port 46 that connects the lumen of the inner tube 24 with an aspiration pump for aspirating tissue, fluid or other debris out of an eye. In other embodiments, the center port 46 may also provide for the infusion of fluid, such as a fluidic substance, into the eye through the lumen of the inner tube 24.

In some embodiments wherein the housing 42 and the end piece 44 are separable, the end piece 44 provides a watertight seal to the mixing chamber 41 by rotationally or frictionally coupling to the housing 42. It may be advantageous for the end piece to be separable from the housing so that various models of the end piece with different numbers of ports may be selectively coupled to the same housing according to varying surgical needs. In other embodiments, the housing 42 and the end piece 44 are integrated into a one-piece unite.

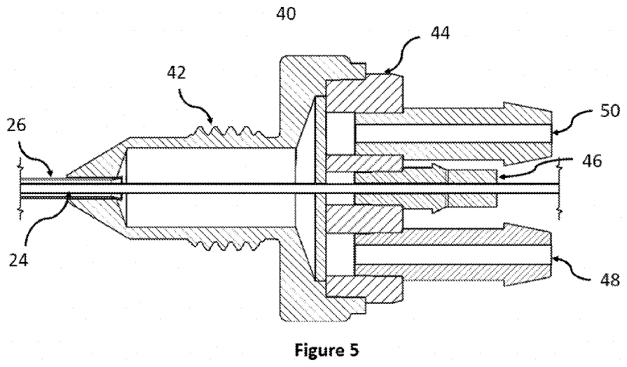

FIG. 5 is a sectional view of the fluidics control 40 according to a second embodiment. In such embodiments, the end piece 44 includes more than one ports for connecting to variable substance chambers. For example, the inlet ports 48 and 50 may be connected to two different substance chambers. The ports 48 and 50 are both fluidically connected to the mixing chamber 41 to enable a surgeon to mix two fluids to produce one or more concentrations of a drug and/or a mixture of the two substances. It is also contemplated that the end piece 44 may include more than two inlet ports for connecting to variable substance chambers.

In some embodiments, the microsurgical device may be used together with a wound sealing sleeve to restrict fluid ingress or egress at the incision wound of the operating site while allowing for proper infusion and/or aspiration. The wound sealing sleeve has a hollow body comprising a conical receiving area, a tubular passage and a tapering section. The conical receiving area has an open end by which the sleeve is attachable to a microsurgical device and the tapering section has an open tip through which a shaft of the device is passed. One or more optional ports or pathways are formed in the sleeve to provide additional source of fluidics transfer in or out of the eye. The wound sealing sleeve could be affixed to the device through threads, a friction fit, glue, or mechanically fastened.

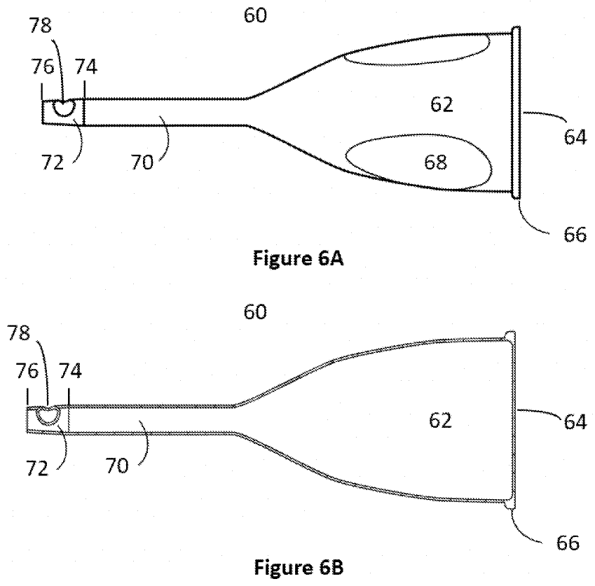

FIG. 6A shows a top view of an exemplary wound sealing sleeve 60 and FIG. 6B is a sectional view of the wound sealing sleeve 60 shown in FIG. 6A. The wound sealing sleeve 60 comprises a receiving area 62 with a receiving space, which is conical in shape, for example, opens out at the proximal end 64 and is surrounded there by a fluidics control flange 66 that extends farther out than the rest of the wall of the receiving area 62. The receiving area 62 may further include one or more flats 68 in an oval shape or other shapes that could be used for finger grips. The receiving area 62 is connected to a tubular passage 70. The tubular passage 70 terminates at a tapering section 72 which serves as an insertion aid. A curved or tapered shoulder extends from the proximal end 74 of the tapering section 72 to the distal open end 76 of the sleeve 60. The distal open end 76 of the sleeve 60 is in sealing contact with the outer surface of the outer tube 26. One or more ports 78 are positioned on the tapering section 72. The depiction of the wound sealing sleeve 60 is for illustrative purposes, understanding that such sleeves are available in a number of different sizes and configurations.

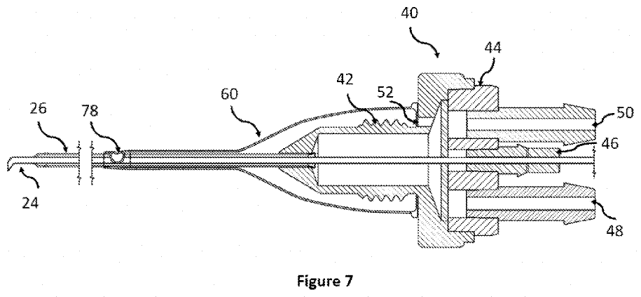

FIG. 7 depicts a sectional view of a fluidics control according to a third embodiment wherein a surgical device is encased by a wound sealing sleeve 60. The fluidics control 40 is received in the receiving area of the wound sealing sleeve 60. It is contemplated that the wound sealing sleeve may be connected to the handle by a fluid tight connection, including threads, a friction fit, glue, or mechanically fastened. Preferably, the wound sealing sleeve may be disposable and rotationally or frictionally connected to the fluidics control 40. The wound sealing sleeve encases at least a portion of the fluidics control 40 and a portion of the shaft. The outer tube 26 of the surgical device extends through the tubular passage 70 of the wound sealing sleeve 60 and protrudes out of the wound sealing sleeve 60 and is thus available with its distal member 14 for the operation. The tubular passage 70 is radially spaced from the outer tube 26.

In some embodiments, a port 50 disposed in the end piece 44 may be fluidically connected to the wound sealing sleeve 60 through an opening 52 in the housing. In such embodiments, the irrigation fluid flows from a substance chamber through the port 50, the opening 52, and the space between the outer surface of the outer tube 26 and the interior wall of the wound sealing sleeve 60, and then out of the port 78 of the wound sealing sleeve 60. It is further contemplated that the fluidics control 40 may include more than one ports that are fluidically connected to the would sealing sleeve 60.

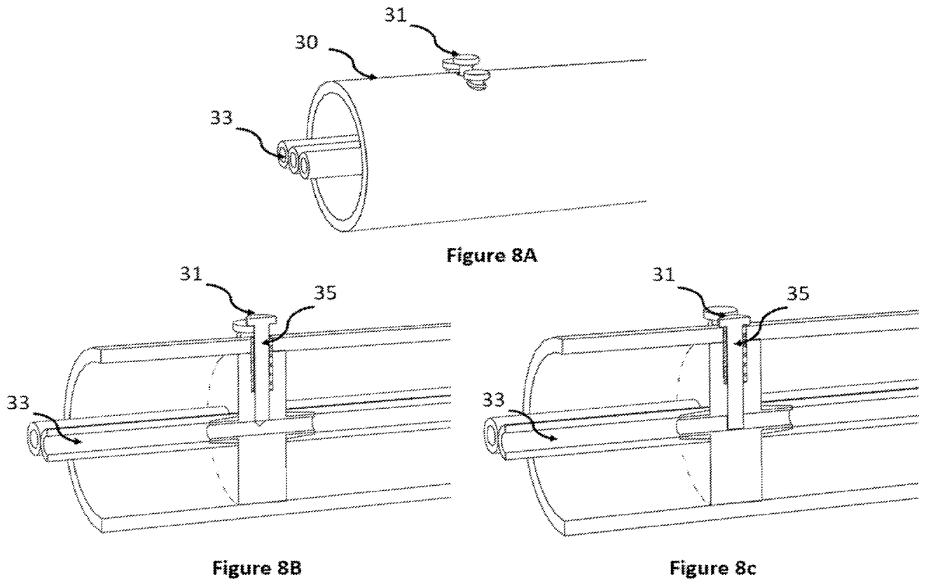

The handheld microsurgical device may further include a fluid control element configured to regulate the fluid flow into or out of the device, or a combination thereof. FIG. 8A depicts a perspective view of an exemplary control element that enable on/off, variable speed, and forward/reverse fluid flow controls. In one exemplary embodiment, the flow is controlled by a user interface mounted on the handle 30. The user interface includes one or more tactile and intuitive switches 31. Suitable switches may include push buttons, joysticks, sliders, or threaded toggles. Each switch may control one single fluid flow path defined by a tubular connector 33. It is also contemplated that one switch may control more than one fluid flow paths.

FIG. 8B is a sectional view of an exemplary switch in an open position and FIG. 8C is a sectional view of an exemplary switch in a closed position. The switch may activate a flow control valve. The valve for controlling the flow of fluid through a connector is situated within the body of the handle. Functions of the valve may include stopping or starting flow, reducing or increasing flow, controlling the direction of flow, and regulating the amount of flow. A variety of flow characteristics can be provided by manufacturing the flow control valves with different configurations.

In one embodiment, the valve is constructed to provide an occluding means. As shown in FIGS. 8B and 8C, this occluding means includes a plug insertable into a connector 33 to stop or start the flow. When the switch 31 activates the valve 35 to be in an open position, as shown in FIG. 8B, the switch 31 permits fluid flow through a tubular connector 33, which is fluidly coupled to the fluidics control 40 at the distal end of the handle 30 to enable transfer of fluid or tissues into or out of the eye. In contrast, when the valve 35 is in a closed position to occlude the connector 33, as shown in FIG. 8C, the switch 31 completely restricts the fluid flow into or out of the fluidics control. In such closed position, the fluid substance may be prevented from being accidentally dispensed from the device, such as by pressure inadvertently applied to the plunger of a substance chamber loaded with the fluid substance.

Preferably, the fluid control element is configured such that the flow of the fluid into or out of the device is controllably variable to a selected rate. For example, the fluid control element may be configured to allow a degree of fluid flow variable between any position in a range of from a fully open position to a fully closed position. In another example, the fluid control element may be configured to provide a variable degree and variable function of fluid flow. A preferred configuration of a fluid control element may provide a fully open position, a fully closed position, and one or more intermediate positions in between, for enabling variable control over a fluid flow.

It is contemplated that the fluid control element may control one or more fluid flow paths simultaneously through a valve assembly. For example, the aspiration port or irrigation port of the fluidics control may be both in fluid communication with respective tubular connectors in no time when the valve assembly is in either the open or closed position. This arrangement is highly beneficial in that each fluid path is operatively switched between open and close, or aspiration and irrigation, thereby enabling simultaneous or multiple aspiration and irrigation operations. This arrangement also enables mixing two or more fluids to produce various concentrations of a drug and/or a mixture of the two or more substances.

It is intended that all matter contained in the above description or shown in the accompanying drawings shall be interpreted as illustrative only and not limiting. Changes in detail or structure may be made without departing from the spirit of the disclosure as defined in the appended claims.

1. A microsurgical device comprising:
a handle;
a shaft having a distal portion that is insertable into an opening of an operating site of an eye, wherein the shaft comprises an outer tube and an inner tube disposed therein, wherein the inner tube extends through a lumen of the outer tube and a distal portion of the inner tube extends out of and beyond a distal end of the outer tube;
a distal member on a distal end of the shaft; and
a fluidics control configured to enable bi-directional flow and fluid separation or mixing;
wherein the fluidics control comprises a housing and an end piece;
wherein the housing comprises a mixing chamber configured to mix multiple fluids or drugs or dilute a concentrated drug to provide varying drug concentration; and
wherein the end piece comprises one or more ports that are connected to an aspiration pump and/or one or more substance chambers.

2. The device of claim 1, further comprising a control element configured to enable selective control of fluids.

3. The device of claim 2, wherein the control element is configured to allow for bolus or continuous delivery.

4. The device of claim 2, wherein the control element is configured to enable a measured delivery of drug at independent rates.

5. The device of claim 2, wherein the control element comprises a user interface mounted on the handle.

6. The device of claim 5, wherein the user interface comprises one or more tactile and intuitive switches.

7. The device of claim 6, wherein the switch activates a flow control valve situated within the handle.

8. The device of claim 7, wherein the switch provides a fully open position, a fully closed position, and one or more intermediate positions for enabling variable control over a fluid flow.

9. The device of claim 1, wherein the handle comprises a body and a lumen formed in the body.

10. The device of claim 1, wherein the handle is in fluid communication with one or more substance chambers.

11. The device of claim 1, wherein the handle is in communication with an aspiration pump.

12. The device of claim 1, wherein the housing and the end piece are separable.

13. The device of claim 12, wherein the end piece is rotationally or frictionally connected to the housing and partially enclosed by the housing.

14. The device of claim 1, wherein the housing and the end piece are in one piece.

15. The device of claim 9, wherein the fluidics control is disposed partially within the body of the handle.

16. The device of claim 15, wherein the housing of the fluidics control is rotationally or frictionally affixed to the body of the handle.

17. The device of claim 1, wherein the inner tube of the shaft has a fluid connection with the housing.

18. The device of claim 1, wherein the inner tube of the shaft bypasses the housing and is fluidically connected to a port that is coupled to the aspiration pump.

19. The device of claim 1, wherein the outer tube of the shaft has a fluid connection with the housing.

20. The device of claim 1, further comprising a sleeve.

21. The device of claim 20, wherein a port in the end piece is fluidically communicated with the sleeve.
